(19) United States
(12) Patent Application Publication
(10) Pub. No.: US 2026/0256362 A1
(43) Pub. Date: Sep. 3, 2026

(54) HEART SOUNDS DATA COLLECTION

(71) Applicant: Cardiac Pacemakers, Inc., St Paul, MN (US)

(72) Inventors: Jeffrey S. Rotter, Rochester, MN (US); Brian Kronstedt, Edina, MN (US); Anand Shroff, Minneapolis, MN (US); David L. Perschbacher, Blaine, MN (US); Keith L. Herrmann, Minneapolis, MN (US)

(21) Appl. No.: 19/553,642

(22) Filed: Mar. 2, 2026

Related U.S. Application Data
(60) Provisional application No. 63/766,106, filed on Mar. 3, 2025.

(51) Int. Cl. A61B 5/0205; A61B 5/00; A61B 5/319; A61B 7/00

(52) U.S. Cl. CPC A61B 5/0205; A61B 5/319; A61B 5/6833; A61B 5/7475; A61B 7/00; A61B 2562/0219

(57) ABSTRACT

Systems, methods, and devices involve approaches for detecting a first set of acceleration data collected at a first sampling frequency; determining that the first set of acceleration data contains noise less than a threshold; and detecting a second set of acceleration data collected at a second sampling frequency that is higher than the first sampling frequency.

### CROSS REFERENCE TO RELATED APPLICATION

This application claims priority to Provisional Application No. 63/766,106, filed Mar. 3, 2025, which is herein incorporated in its entirety.

### TECHNICAL FIELD

The present disclosure relates to devices, methods, and systems for a collecting cardiac activity data.

### BACKGROUND

Cardiac activity monitors can be used to collect and provide physiological data to medical professionals for analysis.

### SUMMARY

In Example 1, a method includes detecting a first set of acceleration data collected at a first sampling frequency; determining that the first set of acceleration data contains noise less than a threshold; and detecting a second set of acceleration data collected at a second sampling frequency that is higher than the first sampling frequency. In certain instances, the detecting is performed only after the determining that the first set of acceleration data contains noise less than a threshold.

In Example 2, the method of Example 1, wherein the first sampling frequency is a multiple of the second sampling frequency.

In Example 3, the method of Examples 1 or 2, wherein the first sampling frequency is 10–100 Hz.

In Example 4, the method of any of Examples 1–3, wherein the detecting the first set of acceleration data occurs periodically on a predetermined schedule.

In Example 5, the method of any of Examples 1–4, wherein the detecting the second set of acceleration data occurs for a predetermined amount of time.

In Example 6, the method of any of Examples 1–5, further including calculating a sensor rectified average of the first set of acceleration data and comparing the SRA to the threshold.

In Example 7, the method of any of Examples 1–6, wherein the detecting the second set of acceleration data occurs in response to detecting a potential cardiac event based, at least in part, on a user input or electrocardiogram (ECG) data.

In Example 8, the method of Example 7, wherein the ECG data is sampled at the second sampling frequency.

In Example 9, the method of Example 7 or 8, wherein the ECG data is sensed by electrodes on a cardiac monitor device, wherein the first set of acceleration data is sensed by a single acceleration sensor on the cardiac monitor device, wherein the second first set of acceleration data is sensed by the single acceleration sensor.

In Example 10, the method of Example 9, wherein the user input is pressing a button on the cardiac monitor device.

In Example 11, the method of any of Examples 1–10, wherein the detecting the first set of acceleration data occurs only after determining that a first housing section is connected to a second housing section.

In Example 12, the method of any of Examples 1–11, further including generating heart sound data using the second set of acceleration data.

In Example 13, a computer program product comprising instructions to cause one or more processors to carry out the steps of the method of Examples 1–12.

In Example 14, a computer-readable medium having stored thereon the computer program product of Example 13.

In Example 15, a monitor comprising the computer-readable medium of Example 14.

In Example 16, a monitor includes a housing comprising an acceleration sensor and circuitry. The circuitry is programmed to: detect, using the acceleration sensor, a first set of acceleration data collected at a first sampling frequency; determine that the first set of acceleration data contains noise less than a threshold; and detect, using the acceleration sensor, a second set of acceleration data collected at a second sampling frequency that is higher than the first sampling frequency.

In Example 17, the monitor of Example 16, wherein the first sampling frequency is a multiple of the second sampling frequency.

In Example 18, the monitor of Example 16, wherein the first sampling frequency is 10–100 Hz.

In Example 19, the monitor of Example 16, wherein the noise is based, at least in part, on a sensor rectified average.

In Example 20, the monitor of Example 16, wherein the circuitry is further programmed to detect the second set of acceleration data in response to detection of a potential cardiac event based, at least in part, on a user input or ECG data.

In Example 21, the monitor of Example 20, wherein the ECG data is sampled at the second sampling frequency.

In Example 22, the monitor of Example 20, wherein the housing is attached to a patch, wherein the patch includes electrodes configured to sense cardiac activation signals used to generate the ECG data.

In Example 23, the monitor of Example 22, wherein monitor includes only one acceleration sensor.

In Example 24, the monitor of Example 20, further including a button, wherein the user input is detected in response to a pressing of the button.

In Example 25, the monitor of Example 16, wherein the housing includes a first housing section and a second housing section.

In Example 26, the monitor of Example 25, wherein the detecting the first set of acceleration data occurs only after determining that a first housing section is connected to a second housing section.

In Example 27, a method includes detecting a first set of acceleration data collected at a first sampling frequency; determining that the first set of acceleration data contains noise less than a threshold; and only after the determining, detecting a second set of acceleration data collected at a second sampling frequency that is higher than the first sampling frequency.

In Example 28, the method of Example 27, wherein the first sampling frequency is a multiple of the second sampling frequency.

In Example 29, the method of Example 27, wherein the first sampling frequency is 10–100 Hz.

In Example 30, the method of Example 27, further comprising calculating an SRA of the first set of acceleration data and comparing the SRA to the threshold.

In Example 31, the method of Example 27, wherein the detecting the second set of acceleration data occurs in response to detecting a potential cardiac event based, at least in part, on a user input or ECG data.

In Example 32, the method of Example 31, wherein the ECG data is sampled at the second sampling frequency.

In Example 33, the method of Example 31, wherein the ECG data is sensed by electrodes on a cardiac monitor device, wherein the first set of acceleration data is sensed by a single acceleration sensor on the cardiac monitor device, wherein the second first set of acceleration data is sensed by the single acceleration sensor.

In Example 34, the method of Example 27, wherein the detecting the first set of acceleration data occurs only after determining that a first housing section is connected to a second housing section.

In Example 35, the method of Example 27, further including generating heart sound data using the second set of acceleration data.

In Example 36, a method includes detecting whether a monitor is connected to a patch; after detecting that the monitor is connected to the patch, detecting a first set of acceleration data collected at a first sampling frequency; and detecting a second set of acceleration data collected at a second sampling frequency that is higher than the first sampling frequency in response to detecting a potential cardiac event. In certain instances, the second set of acceleration data is collected only if noise in the first set of acceleration data is below a noise threshold level.

In Example 37, a method includes detecting a potential cardiac event; and, in response to detecting the potential cardiac event, detecting heart sounds data using an acceleration sensor. In certain instances, the heart sounds data is collected only if an estimated noise from the acceleration sensor data is below a noise threshold level. In certain instances, the estimated noise is based on acceleration data that is collected at a lower sampling frequency than the heart sounds data.

While multiple instances are disclosed, still other instances of the present disclosure will become apparent to those skilled in the art from the following detailed description, which shows and describes illustrative instances of the disclosure. Accordingly, the drawings and detailed description are to be regarded as illustrative in nature and not restrictive.

### BRIEF DESCRIPTION OF THE DRAWINGS

FIG. 1 shows a cardiac data monitoring system, in accordance with certain instances of the present disclosure.

FIG. 2 shows a cardiac monitor, in accordance with certain instances of the present disclosure.

FIG. 3 shows a housing of the cardiac monitor of FIG. 2, in accordance with certain instances of the present disclosure.

FIGS. 4–7 show diagrams outlining approaches for conserving battery power, in accordance with certain instances of the present disclosure.

FIG. 8 is a block diagram depicting an illustrative computing device, in accordance with instances of the disclosure.

While the disclosed subject matter is amenable to various modifications and alternative forms, specific instances have been shown by way of example in the drawings and are described in detail below. The intention, however, is not to limit the disclosure to the particular instances described. On the contrary, the disclosure is intended to cover all modifications, equivalents, and alternatives falling within the scope of the disclosure as defined by the appended claims.

### DETAILED DESCRIPTION

The present disclosure relates to cardiac activity monitors (hereinafter “monitors” for brevity) and methods of using such monitors. A monitor can be used to sense and collect various cardiac activity data such as electrocardiogram (“ECG”) data and/or heart sounds data.

One example type of monitor is a wearable patch-based monitor that is attached to a patient. To collect ECG data, the monitor can include electrodes that sense a patient’s cardiac electric activation signals and generate responsive ECG data. To collect heart sounds data, the monitor can include one or more acceleration sensors (e.g., accelerometers) that sense acoustic signals generated by a patient’s heart. ECG data and heart sounds data can be analyzed to determine whether the patient has experienced a cardiac event and details about the cardiac event, among other things.

Monitors are typically battery powered. Battery capacity can be limited due to overall size limitations of monitors (and therefore size limitations of the battery). Continuously sensing and storing ECG data and heart sounds data can be relatively power-intensive given the battery constraints of monitors. Collecting heart sounds data in particular can be a relatively power-intensive process.

Instances of the present disclosure utilize approaches that help conserve battery power of monitors.

### CARDIAC DATA MONITORING SYSTEM

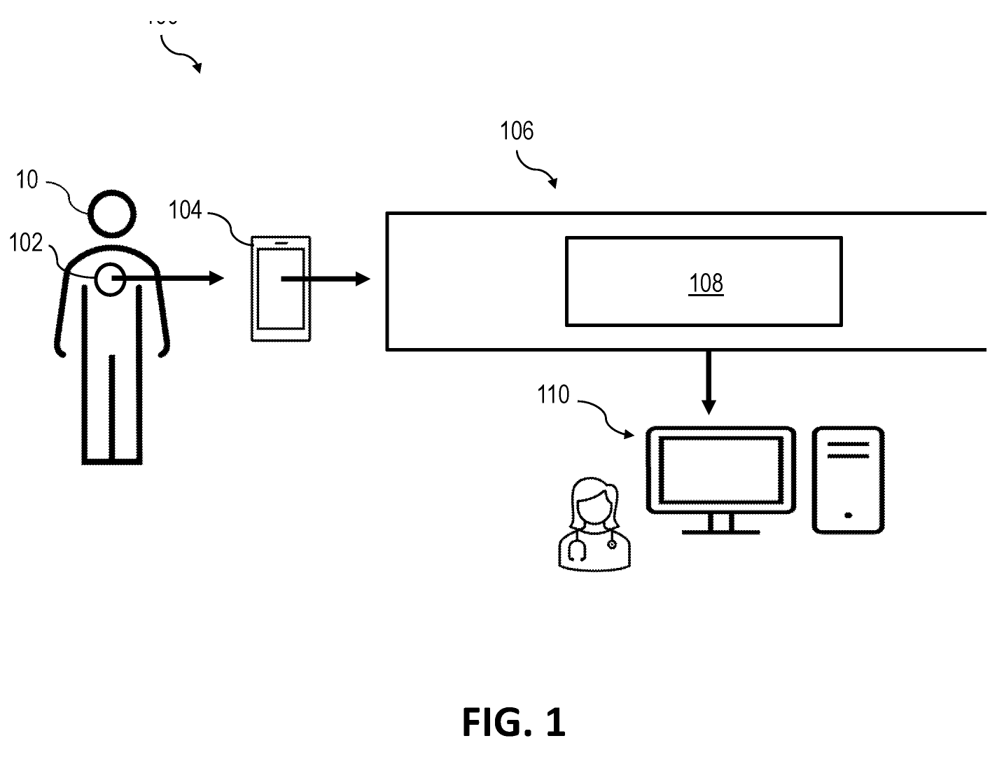

FIG. 1 illustrates a patient 10 and an example system 100. The system 100 includes a monitor 102 used to detect cardiac activity of the patient 10. The monitor 102 can be, for example, an implantable medical device or a wearable medical device that includes one or more sensing devices (e.g., electrodes that sense a patient’s cardiac electric activation signals; sensors such as accelerometers that sense acoustic signals generated by the heart) and generate cardiac activity data such as ECG data and heart sounds data.

In some instances, the monitor 102 itself processes and analyzes at least some of the cardiac activity data. For example, the monitor 102 can be programmed to detect potential cardiac events based on the cardiac activity data (or a subset thereof). However, more power-intensive processes can be performed by separate devices/systems.

Cardiac activity data can be offloaded to an external computing device for additional processing and analysis. For example, the monitor 102 can transmit the cardiac activity data (including associated metadata) to an intermediate computing device 104 such as a mobile device (e.g., a smartphone), a computer, a server, a laptop, etc. which then transmits at least some of the cardiac activity data to a separate server 106. As another example, the monitor 102 transmits the cardiac activity data to the server 106 without using an intermediate computing device.

The server 106 can include platforms, layers, or modules that work together to process and analyze the cardiac activity data such that cardiac events and activity can be detected, filtered, prioritized, and ultimately reported to a patient’s physician for analysis, diagnosis, and treatment, if needed. In the example of FIG. 1, the server 106 includes one or more machine learning models 108 (e.g., types of deep neural networks). The server 106 applies the one or more machine learning models 108 to the cardiac activity data to classify cardiac activity of the patient 10. For example, for ECG data, the machine learning model 108 may compare ECG data collected by the monitor 102 to labeled ECG data to determine which labeled ECG data the ECG data most closely resembles. The labeled ECG data may identify a particular cardiac event, including but not limited to ventricular tachycardia, bradycardia, atrial fibrillation, pause, normal sinus rhythm, artifact/noise, etc. Further, the labeled ECG data may identify beat classifications such as normal, ventricular, and supraventricular.

If the machine learning model 108 determines that the ECG data most closely resembles a labeled ECG data associated with a cardiac event, then the machine learning model 108 may determine that the patient 10 has experienced that cardiac event. Additionally, the machine learning model 108 may measure or determine certain characteristics of the cardiac activity of the patient 10 based on the ECG data. For example, the machine learning model 108 may determine a heart rate, a duration, or a beat count of the patient 10 during the cardiac event based on the ECG data. The server 106 stores the cardiac event (and associated metadata such as information like beat classification, heart rate, duration, beat count, etc.) in a database for storage. Certain outputs of the server 106 can be accessed by approved users via a remote computer 110 (e.g., client device such as a laptop, mobile phone, desktop computer, and the like) by a user at a clinic or laboratory.

The server 106 can also be programmed to process and analyze heart sounds data.

### WEARABLE CARDIAC ACTIVITY MONITOR

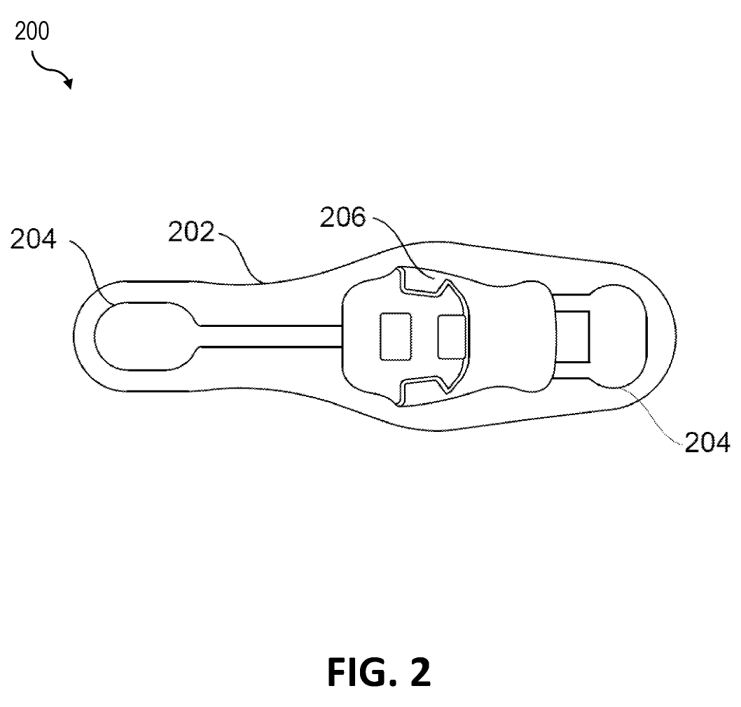

FIG. 2 shows parts of a monitor 200, including a patch 202 is designed to be directly attached to skin of a patient. As described herein, the monitor 200 can be used to detect physiological data such as ECG data and heart sounds data.

In the example of FIG. 2, the patch 202 includes electrodes 204 that are configured to sense cardiac activity data such that ECG data can be generated. The electrodes 204 can include a hydrogel layer (which is coupled to the patient’s skin) and a conductive metal layer, wire, etc., that communicates sensed signals to circuitry (e.g., integrated circuitry such as amplifiers, processors, and the like) which processes and analyzes ECG data.

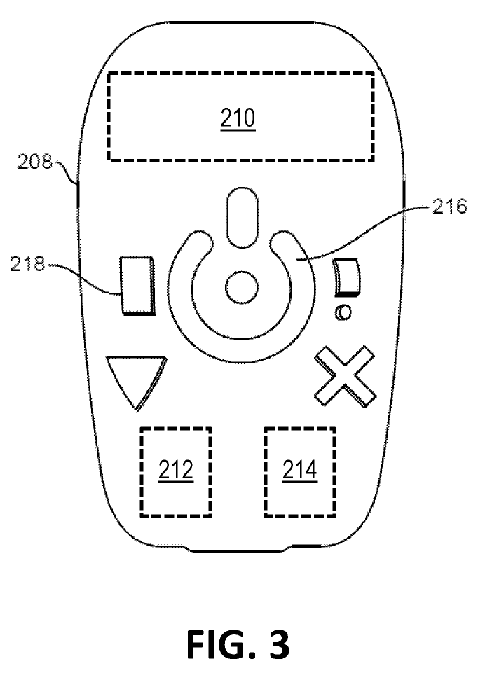

A first housing section 206 is attached to the patch 202 and can include features that allow a second housing section (shown in FIG. 3) to be coupled and decoupled to the first housing section 206. As one example, the first housing section 206 can include a cavity in which the second housing section is inserted. As another example, the first housing section 206 can include mechanical features such as tabs, clips, etc., that couple to features of the second housing section to temporarily, mechanically couple the two housing sections together. The first housing section 206 can also include an electrical connector to facilitate electrical connection with the electrodes 204 of the patch 202.

FIG. 3 shows a second housing section 208 of the monitor 200. The second housing section 208 can be a removable housing section that can be removably coupled to the first housing section 206. The second housing section 208 can include a battery 210 (e.g., a rechargeable battery) that powers circuitry 212, which processes, stores, and analyzes the signals measured by the electrodes 204.

The second housing section 208 can include one or more sensors 214 such as an acceleration sensor. The acceleration sensor can be used to detect heart sounds. Heart sounds are created by the opening and closing of heart valves — as well as the flow of blood through the heart — which produce acoustic signals. Heart sounds may be measured and used, for example, to indicate the heart’s mechanical activities. A heart sound can include audible and inaudible mechanical vibrations caused by cardiac activity that can be sensed with an acceleration sensor.

There are different clinical categories of heart sounds. For example, the S1 heart sound refers to the first heart sound of a cardiac cycle, S2 refers to the second heart sound, S3 refers to the third heart sound, and S4 refers to the fourth heart sound. S1 is known to be indicative of, among other things, mitral valve closure, tricuspid valve closure, and aortic valve opening. S2 is known to be indicative of, among other things, aortic valve closure and pulmonary valve closure. S3 is known to be a ventricular diastolic filling sound often indicative of certain pathological conditions including heart failure. And S4 is known to be a ventricular diastolic filling sound resulted from atrial contraction and is usually indicative of pathological conditions.

In certain instances, the acceleration sensor is a 1-D accelerometer or a multi-axis accelerometer. The acceleration sensor can be positioned on the monitor 200 such that acceleration along an axis pointed away from a person’s chest is sensed by the acceleration sensor.

The second housing section 208 can also include buttons 216 (e.g., pressable buttons) such as a power button, an event button, etc. The event button can be pressed by the patient when the patient believes they are experiencing symptoms of a cardiac event (e.g., increased heart rate, arrhythmia). The second housing section 208 can also include indicators 218 such as battery-charge indicators, power indicators, etc.

As noted above, battery capacity can be limited due to overall size limitations of monitors. Instances of the present disclosure assist utilize approaches that help conserve battery power of monitors.

### POWER CONSERVATION APPROACHES

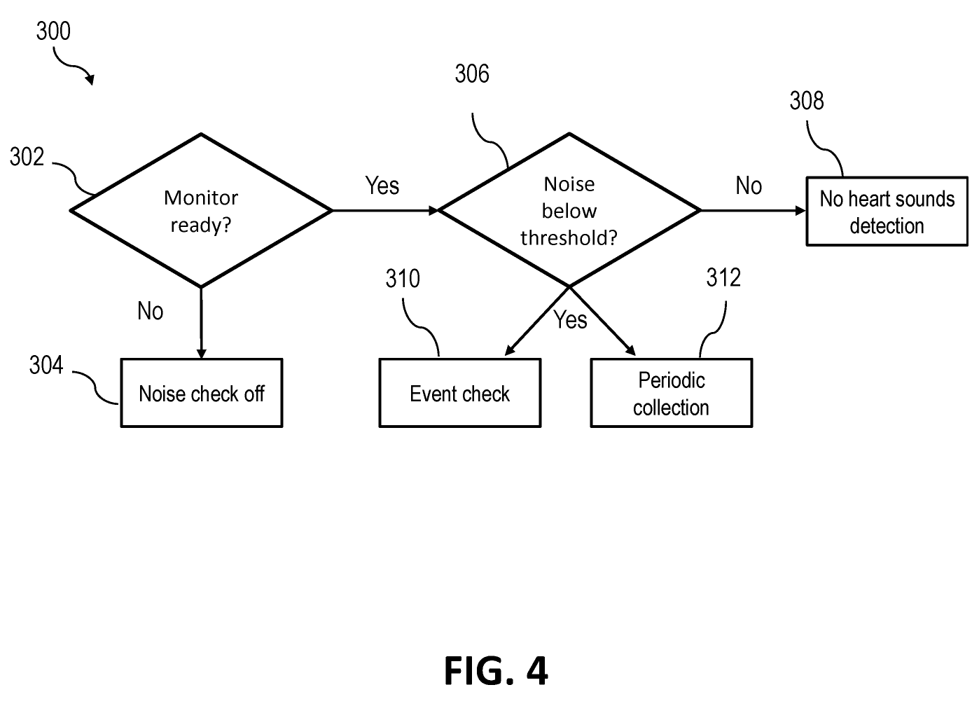

FIG. 4 shows a flowchart 300 that outlines various approaches for conserving battery power of cardiac activity monitors such as the monitors of FIGS. 1–3. In certain instances, the approaches can be used to collect heart sounds data periodically (as opposed to continuously) and/or only when certain conditions are met. As a result, battery power usage can be reduced because heart sounds data is collected (and therefore stored and transmitted) less often.

In certain instances, the flowchart 300 begins by determining whether the monitor is ready for detecting cardiac activity (block 302). For monitors constructed like the monitor 200 of FIGS. 2 and 3, this determination can be based on whether the second housing section 208 is connected to the first housing section 206. For example, the monitor can determine that the two housing sections are connected by detecting whether conductive members (e.g., conductive pins, pads, and the like) are conductively coupled to each other. Each housing section can include a conductive member that is arranged to contact a corresponding conductive member of the other housing section. When the conductive members are coupled to each other, the monitor can detect the coupling. In some instances, connecting the first housing section to the second housing section changes a resistor value. In certain instances, the resistor value also indicates which type of device (e.g., patch, lead set) the monitor is connected to.

If the monitor is not ready for detecting cardiac activity, the monitor can be operated in a low-power-consumption mode, where certain operations are not turned on. In this mode, algorithms such as cardiac event detection algorithms and noise check algorithms are not turned on (block 304). Algorithms are discussed further herein.

If the monitor is ready for detecting cardiac activity, the monitor can operate various algorithms such as those that collect and analyze acceleration data and/or physiological data such as ECG data.

In certain instances, acceleration data is used as part of a noise check algorithm (block 306). In short, the noise check algorithm uses acceleration data collected at a lower sampling rate to estimate whether collecting higher-sampling-rate acceleration data will result in useful heart sounds data that can be successfully processed and analyzed (e.g., acceleration data that contains an acceptable level of noise). This can conserve battery power by not collecting heart sounds data when it is unlikely to result in analyzable heart sounds data. Noise in acceleration data can be caused by physical activity of the user such as walking, exercising, moving, etc.

The acceleration data for the noise check algorithm can include a first set of acceleration data that is collected by an acceleration sensor (e.g., an accelerometer) at a first sampling frequency. In certain instances, the first sampling frequency is 10–100 Hz such as 25 Hz. Noise in the first set of acceleration data can be estimated using various approaches, such as using a sensor rectified average (SRA) calculation, a root mean square (RMS) calculation, a frequency domain-based analysis using a fast fourier transform (FFT), and the like.

The estimated noise in the first set of acceleration data can be compared to a threshold noise level (e.g., an SNR value, an absolute value) to determine whether the first set of acceleration data contains noise less than or more than the threshold noise level (block 306). If the estimated noise is above the threshold noise level, then the monitor can continue to collect acceleration data at the lower sampling rate to conserve battery power (block 308). As a result, the monitor will not collect heart sounds data if there is too much estimated noise (as set by the threshold noise level) in the lower-sampling-rate acceleration data.

If the estimated noise is below the threshold noise level, then the monitor can utilize various operations that collect higher-sampling-rate acceleration data.

In one operation, a second set of acceleration data (at a higher sampling rate) is collected in response to detecting a potential cardiac event (block 310). The potential cardiac event can be based on, at least in part, a user input or physiological data such as ECG data.

The user input can be the act of the user pressing/selecting a button, icon, etc. As one example, a button can be a pressable button on the monitor such as the button 216 on the monitor 200 of FIG. 3. As another example, an icon can be part of a graphical user interface on a mobile computing device such as a smartphone operating a cardiac monitoring application.

Additionally or alternatively, the potential cardiac event of block 310 can be based on ECG data collected by the monitor. The monitor can include electrodes that are attached to the user, and the signals sensed by the electrodes can be processed and analyzed by circuitry of the monitor. The monitor can be programmed to detect cardiac events such as tachycardia, bradycardia, atrial fibrillation, pause, etc. These cardiac events may be detected based on one or more thresholds (e.g., heart rate, average heart rate, RR interval) utilized by the circuitry of the monitor.

After a potential cardiac event is detected, acceleration data indicative of heart sounds data can begin to be collected (e.g., via an acceleration sensor of the monitor). The acceleration data can be collected at a higher sampling frequency than the sampling frequency of the acceleration data used for the noise check algorithm. In certain instances, the higher sampling frequency is a multiple of the lower sampling frequency. For example, if the lower sampling frequency is 10–100 Hz and the multiple is “10”, the higher sampling frequency can be 100–1,000 Hz such as 250 Hz when the lower sampling frequency is 25 Hz. As a result, the same acceleration sensor can be used for both sets of acceleration data.

In certain instances, the ECG data collected by the monitor is sampled at the same sampling frequency of the higher-sampling-frequency acceleration data. As a result, the same clock source can be used for both the ECG data and the acceleration data.

Once a potential cardiac event has been detected, the higher-sampling-frequency acceleration data can be collected using the acceleration sensor. In certain instances, the higher-sampling-frequency acceleration data can be collected for a predetermined amount of time. For example, such acceleration data can be collected for a set time after the detection (e.g., 30 seconds to 5 minutes, 30 seconds to 3 minutes, 30 seconds to 2 minutes). The acceleration data can be saved to local memory of the monitor and eventually transmitted to an external computing device. In certain instances, because processing heart sounds data can be a power-intensive process, the monitor may not be programmed to process the heart sounds data. Instead, an external computing device (e.g., a server) that receives the acceleration data can be used to process the heart sounds data such that the data can be viewed and analyzed by a physician.

Because ECG data is collected using electrodes (and not an acceleration sensor), the monitor can be programmed to detect cardiac events regardless of the output of the noise check algorithm. However, if the estimated noise is above the noise threshold level, the acceleration sensor can continue to collect data at the lower sampling rate — even if a potential cardiac event is detected.

Another operation that can be used if the estimated noise is below the threshold noise level, is an operation that periodically collects acceleration data at a higher sampling rate (block 312) — even if no potential cardiac event is detected. For example, the noise check algorithm can be operated periodically (e.g., every 1–10 minutes), and higher-sampling-rate acceleration data can be collected in the event the estimated noise is below the noise threshold level. In certain instances, the higher-sampling-rate acceleration data can be collected for a predetermined length of time such as 30–60 seconds.

As a result of the approaches described herein, collection, storage, and transmission of heart sounds data is reduced compared to approaches that continuously collect heart sounds data (e.g., higher-sampling-rate acceleration data). This reduces battery consumption and therefore can extend the time between needed recharging of the battery of cardiac activity monitors. These approaches also reduce the amount of data that is stored, processed, etc., which reduces the need for larger memory capacity and energy spent storing and processing data — particularly data that may not be useful for analysis and diagnosis.

### METHODS

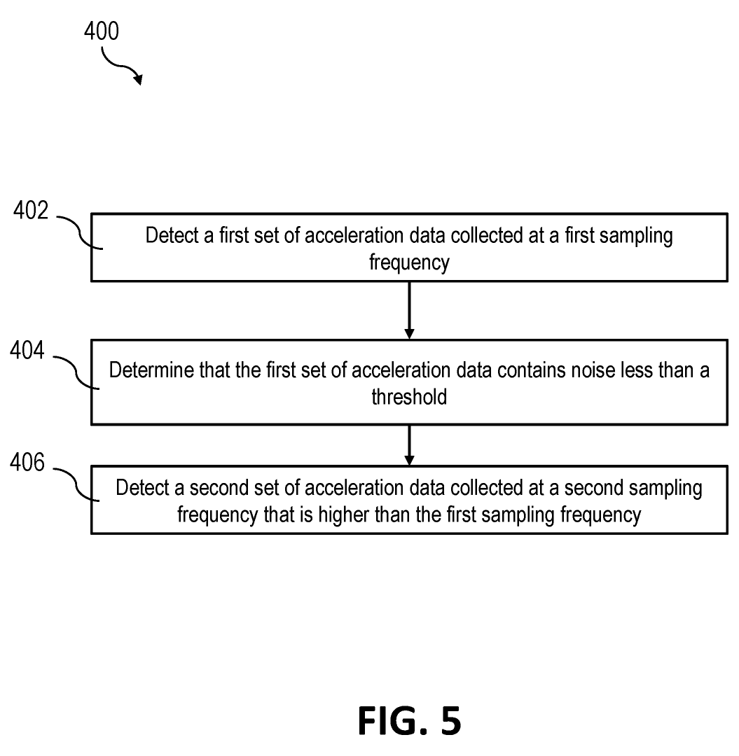

FIG. 5 shows a block diagram of an example method 400 for conserving battery power. The method 400 includes detecting a first set of acceleration data collected at a first sampling frequency (block 402 in FIG. 5). The method 400 further includes determining that the first set of acceleration data contains noise less than a threshold (block 404 in FIG. 5). After determining that the first set of acceleration data contains noise less than a threshold, the method 400 includes detecting a second set of acceleration data collected at a second sampling frequency that is higher than the first sampling frequency (block 406 in FIG. 5).

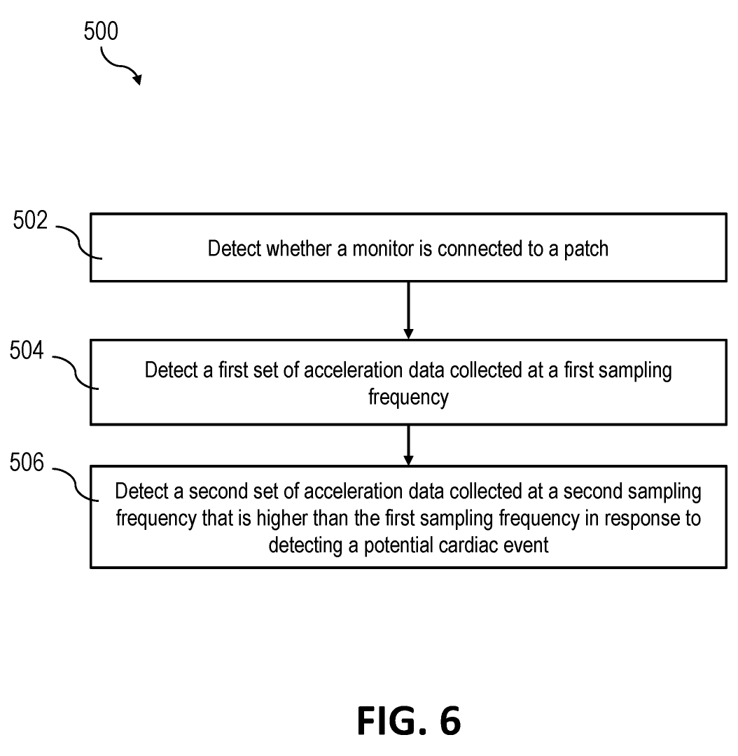

FIG. 6 shows a block diagram of an example method 500 for conserving battery power. The method 500 includes detecting whether a monitor is connected to a patch (e.g., a patch with electrodes for sensing cardiac activation activity) (block 502 in FIG. 6). After detecting that the monitor is connected to the patch, the method 500 includes detecting a first set of acceleration data collected at a first sampling frequency (block 504 in FIG. 5). The method 500 further includes detecting a second set of acceleration data collected at a second sampling frequency that is higher than the first sampling frequency in response to detecting a potential cardiac event (block 506 in FIG. 6). In certain instances, the second set of acceleration data is collected only if noise in the first set of acceleration data is below a noise threshold level.

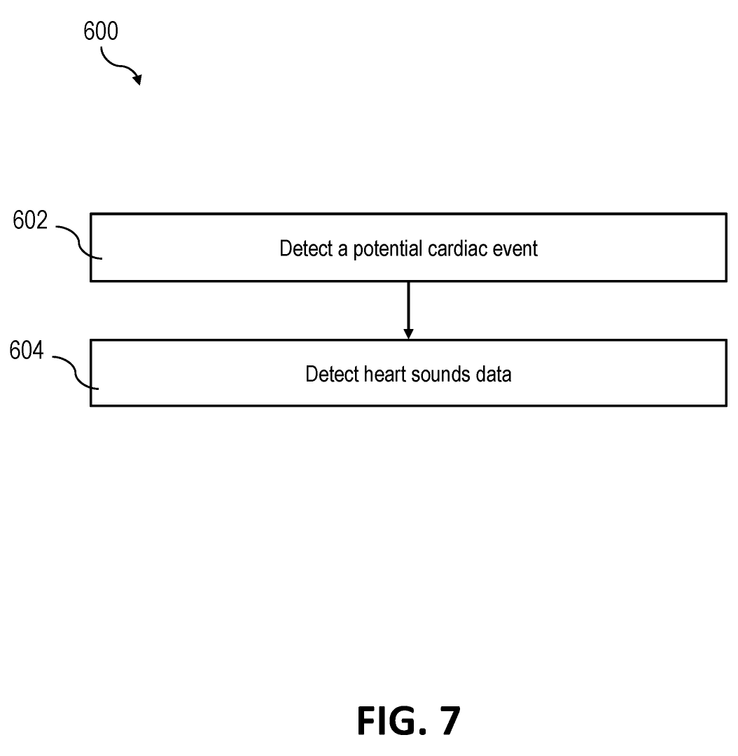

FIG. 7 shows a block diagram of an example method 600 for conserving battery power. The method 600 includes detecting a potential cardiac event (block 602 in FIG. 7) such as by using ECG data. In response to detecting the potential cardiac event, the method 600 includes detecting heart sounds data using an acceleration sensor (block 604 in FIG. 7). In certain instances, the heart sounds data is collected only if an estimated noise from the acceleration sensor data is below a noise threshold level. In certain instances, the estimated noise is based on acceleration data that is collected at a lower sampling frequency than the heart sounds data.

### COMPUTING DEVICES AND SYSTEMS

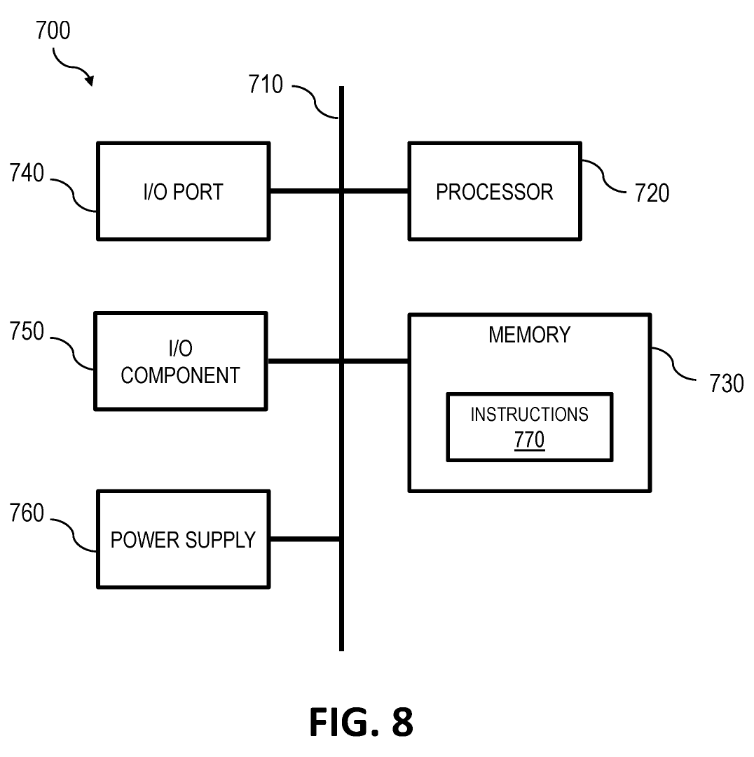

FIG. 8 is a block diagram depicting an illustrative computing device 700, in accordance with instances of the disclosure. The computing device 700 may include any type of computing device suitable for implementing aspects of instances of the disclosed subject matter. Examples of computing devices include specialized computing devices or general-purpose computing devices such as workstations, servers, laptops, desktops, tablet computers, hand-held devices, smartphones, general-purpose graphics processing units (GPGPUs), and the like. Each of the various components shown and described in the Figures can contain their own dedicated set of computing device components shown in FIG. 8 and described below. For example, the cardiac activity monitors, external computing device, and server can each include their own set of components shown in FIG. 8 and described below.

In instances, the computing device 700 includes a bus 710 that, directly and/or indirectly, couples one or more of the following devices: a processor 720, a memory 730, an input/output (I/O) port 740, an I/O component 750, and a power supply 760. Any number of additional components, different components, and/or combinations of components may also be included in the computing device 700.

The bus 710 represents what may be one or more busses (such as, for example, an address bus, data bus, or combination thereof). Similarly, in instances, the computing device 700 may include a number of processors 720, a number of memory components 730, a number of I/O ports 740, a number of I/O components 750, and/or a number of power supplies 760. Additionally, any number of these components, or combinations thereof, may be distributed and/or duplicated across a number of computing devices.

In instances, the memory 730 includes computer-readable media in the form of volatile and/or nonvolatile memory and may be removable, nonremovable, or a combination thereof. Media examples include random access memory (RAM); read only memory (ROM); electronically erasable programmable read only memory (EEPROM); flash memory; optical or holographic media; magnetic cassettes, magnetic tape, magnetic disk storage or other magnetic storage devices; data transmissions; and/or any other medium that can be used to store information and can be accessed by a computing device. In instances, the memory 730 stores computer-executable instructions 770 for causing the processor 720 to implement aspects of instances of components discussed herein and/or to perform aspects of instances of methods and procedures discussed herein. The memory 730 can comprise a non-transitory computer readable medium storin the computer-executable instructions 770.

The computer-executable instructions 770 may include, for example, computer code, machine-useable instructions, and the like such as, for example, program components capable of being executed by one or more processors 720 (e.g., microprocessors) associated with the computing device 700. Program components may be programmed using any number of different programming environments, including various languages, development kits, frameworks, and/or the like. Some or all of the functionality contemplated herein may also, or alternatively, be implemented in hardware and/or firmware.

According to instances, for example, the instructions 770 may be configured to be executed by the processor 720 and, upon execution, to cause the processor 720 to perform certain processes. In certain instances, the processor 720, memory 730, and instructions 770 are part of a controller such as an application specific integrated circuit (ASIC), field-programmable gate array (FPGA), and/or the like. Such devices can be used to carry out the functions and steps described herein.

The I/O component 750 may include a presentation component configured to present information to a user such as, for example, a display device, a speaker, a printing device, and/or the like, and/or an input component such as, for example, a microphone, a joystick, a satellite dish, a scanner, a printer, a wireless device, a keyboard, a pen, a voice input device, a touch input device, a touch-screen device, an interactive display device, a mouse, and/or the like.

The devices and systems described herein can be communicatively coupled via a network, which may include a local area network (LAN), a wide area network (WAN), a cellular data network, via the internet using an internet service provider, and the like.

Aspects of the present disclosure are described with reference to flowchart illustrations and/or block diagrams of methods, devices, systems and computer program products. It will be understood that each block of the flowchart illustrations and/or block diagrams, and combinations of blocks in the flowchart illustrations and/or block diagrams, can be implemented by computer program instructions.

Various modifications and additions can be made to the exemplary instances discussed without departing from the scope of the disclosed subject matter. For example, while the instances described above refer to particular features, the scope of this disclosure also includes instances having different combinations of features and instances that do not include all of the described features. Accordingly, the scope of the disclosed subject matter is intended to embrace all such alternatives, modifications, and variations as fall within the scope of the claims, together with all equivalents thereof.

We claim:

1. A monitor comprising:
a housing comprising an acceleration sensor and circuitry, wherein the circuitry is programmed to:
detect, using the acceleration sensor, a first set of acceleration data collected at a first sampling frequency,
determine that the first set of acceleration data contains noise less than a threshold, and
detect, using the acceleration sensor, a second set of acceleration data collected at a second sampling frequency that is higher than the first sampling frequency.

2. The monitor of claim 1, wherein the first sampling frequency is a multiple of the second sampling frequency.

3. The monitor of claim 1, wherein the first sampling frequency is 10–100 Hz.

4. The monitor of claim 1, wherein the noise is based, at least in part, on a sensor rectified average.

5. The monitor of claim 1, wherein the circuitry is further programmed to detect the second set of acceleration data in response to detection of a potential cardiac event based, at least in part, on a user input or electrocardiogram (ECG) data.

6. The monitor of claim 5, wherein the ECG data is sampled at the second sampling frequency.

7. The monitor of claim 5, wherein the housing is attached to a patch, wherein the patch includes electrodes configured to sense cardiac activation signals used to generate the ECG data.

8. The monitor of claim 7, wherein monitor includes only one acceleration sensor.

9. The monitor of claim 5, further comprising a button, wherein the user input is detected in response to a pressing of the button.

10. The monitor of claim 1, wherein the housing includes a first housing section and a second housing section.

11. The monitor of claim 10, wherein the detecting the first set of acceleration data occurs only after determining that a first housing section is connected to a second housing section.

12. A method comprising:
detecting a first set of acceleration data collected at a first sampling frequency;
determining that the first set of acceleration data contains noise less than a threshold; and
only after the determining, detecting a second set of acceleration data collected at a second sampling frequency that is higher than the first sampling frequency.

13. The method of claim 12, wherein the first sampling frequency is a multiple of the second sampling frequency.

14. The method of claim 12, wherein the first sampling frequency is 10–100 Hz.

15. The method of claim 12, further comprising calculating a sensor rectified average (SRA) of the first set of acceleration data and comparing the SRA to the threshold.

16. The method of claim 12, wherein the detecting the second set of acceleration data occurs in response to detecting a potential cardiac event based, at least in part, on a user input or electrocardiogram (ECG) data.

17. The method of claim 16, wherein the ECG data is sampled at the second sampling frequency.

18. The method of claim 16, wherein the ECG data is sensed by electrodes on a cardiac monitor device, wherein the first set of acceleration data is sensed by a single acceleration sensor on the cardiac monitor device, wherein the second first set of acceleration data is sensed by the single acceleration sensor.

19. The method of claim 17, wherein the detecting the first set of acceleration data occurs only after determining that a first housing section is connected to a second housing section.

20. The method of claim 17, further comprising generating heart sound data using the second set of acceleration data.
